(19) United States
(12) Patent Application Publication
(10) Pub. No.: US 2008/0294261 A1
(43) Pub. Date: Nov. 27, 2008

(54) Method for treating herniated discs

(76) Inventors: Kevin Pauza, Tyler, TX (US); Brian Burkinshaw, Pflugerville, TX (US); James Rogan, Austin, TX (US)

Correspondence Address: ANDREWS KURTH LLP;Intellectual Property Department, Suite 1100, 1350 I Street, NW, Washington, DC, 20005, US

(21) Appl. No.: 11/802,641

(22) Filed: May 24, 2007

(51) Int. Cl. A61F 2/44; A61F 2/28

(52) U.S. Cl. 623/17.16; 623/23.58

(57) ABSTRACT

A method is effective for treating herniated discs. The method includes (a) surgically remove a herniated portion of disc annulus from a herniated disc, or surgically depressing and holding a herniated portion of disc annulus within original boundaries of the herniated disc, and (b) re-enforcing the surgically treated disc with an effective amount of a biocompatible degradable polymeric compound, such as a fibrin sealant.

### BACKGROUND

The vertebral column, or the spinal column, is composed of a series of connected bones called “vertebrae.” The vertebrae surround the spinal cord and protect it from damage. Nerves branch off the spinal cord and travel to the rest of the body, allowing for communication between the brain and the body.

The vertebrae are connected by spongy intervertebral discs and two small joints called “facet” joints. The intervertebral disc, which is made up of strong connective tissues that hold one vertebra to the next, acts as a cushion or shock absorber between the vertebrae. The disc and facet joints allow for movements of the vertebrae.

As shown in FIG. 1, an intervertebral disc is composed of a tough outer layer called the “annulus fibrosus” and a gel-like center called “nucleus pulposus.” The annulus fibrosus is a strong radial tire-like structure made up of lamellae; concentric sheets of collagen fibers connected to the vertebral end plates. The sheets are orientated at various angles. The annulus fibrosus encloses the gel-like nucleus pulposus.

A herniated disc usually is caused by wear and tear of the disc (also called disc degeneration). As people get older, the center of the disc may start to lose water content, making the disc less effective as a cushion. As a disc deteriorates, the annulus fibrosus can also tear. This can allow displacement of the nucleus pulposus through a crack in the annulus fibrosus, into the space occupied by the nerves and spinal cord. The herniated disc can then press on the nerves and cause pain, numbness, tingling or weakness in the extremities.

Treatment for herniated discs includes local injection of anti-inflammatory medications, such as steroids and non-steroid anti-inflammatory drugs (NSAIDs), physical therapy, behavior modification, intradiscal electrothermal therapy (IDET) and surgery. The surgery can be performed as either an open or mini-open surgery, using very small opening incisions or percutaneously, utilizing specially designed instruments and radiographic techniques.

When herniated discs are surgically treated to remove the herniated portion of the disc annulus, relieving pressure on the spinal nerves, the annulus integrity becomes compromised. This will often result in an annulus fibrosis that may re-herniate, or more likely, will leak nucleus pulposus from the nucleus of the disc, through the weakened zone of the annulus fibrosis, onto the nerve complex surrounding and adjacent to the disc. The nucleus pulposus generates a highly inflammatory response around the exposed nerve complex and causes continued discogenic pain. This phenomenon, sometimes called induced leaky disc syndrome, is a common side effect for procedures that remove the herniated portion of the disc annulus.

Accordingly, there still remains a need for improved treatment regimens that are more effective and with less side effects.

### SUMMARY

What is disclosed is a method of treating herniated discs. The method comprises the steps of surgically removing a herniated portion of disc annulus from said herniated disc, and re-enforcing the surgically treated disc with an effective amount of a biocompatible degradable polymeric compound.

Also disclosed is another method of treating a herniated disc. The method comprises the steps of (a) surgically removing a herniated portion of disc annulus from said herniated disc, (b) placing a dart, suture, staple or its equivalent in a disc area weakened by step (a), and (c) re-enforcing the surgically treated disc with an effective amount of a biocompatible degradable polymeric compound.

### DESCRIPTION OF THE DRAWINGS

The detailed description will refer to the following drawings in which:

FIG. 1 is a cross-sectional view of a vertebral body at the disk space exhibiting hernia which may be treated according to the herein disclosed embodiments;

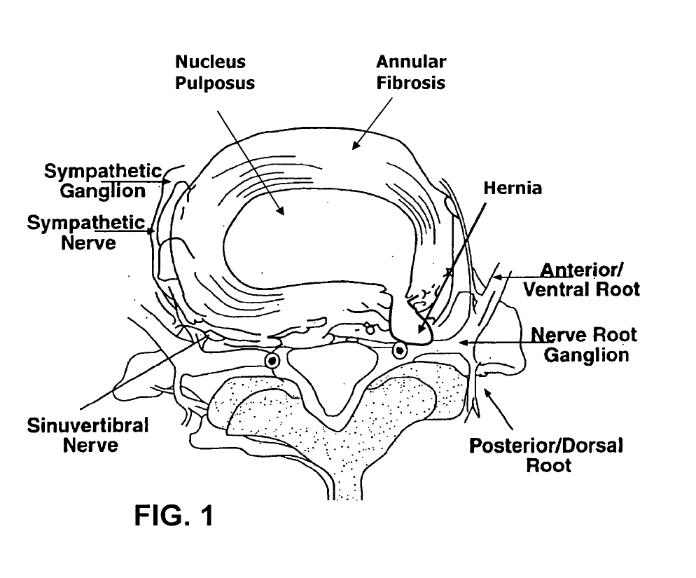

FIGS. 2A-2C are flow charts showing embodiments of the method for treating herniated discs and discogenic pain.

FIG. 3 illustrates an embodiment of a delivery device for injecting fluids into a spinal disc to treat degenerative disc disease and discogenic pain.

FIG. 4 shows pictures of certain commercially available resorbable arrows, darts and staples.

### DETAILED DESCRIPTION

A common side effect of surgical procedures that remove or depress the herniated portion of the disc annulus is a post-operation discogenic pain, which is believed to be caused by the leak age of nucleus pulposus through the area of annulus fibrosis that is weakened by the surgical procedure. A novel method is disclosed to treat a herniated disc by surgically removing or depressing the herniated portion of the herniated disc and then administering a biocompatible degradable polymeric compound, such as a fibrin sealant, to re-enforce the disc areas weakened by the surgical procedure. By injecting (internally and externally) directly into and around the weakened disc annulus with fibrin sealant, a biologic plug is created in the annulus that stops the leakage of nucleus pulposus. The fibrin sealant also provides a natural biologic protective sheath over the exposed nerve endings, and prevents the inflammatory mediators, such as cytokines and proteases, from creating discogenic pain (associated with chemical irritation of nerves around the disc) and/or radicular pain (associated with chemical irritation of the nerve root). Moreover, the fibrin sealant may provide a temporary healing stimulus that signals the body's natural healing mechanisms to act on the site. Finally, the fibrin sealant may act as a buffering agent in the disc, reducing the acidic level of pH, which in turn reduces the inflammatory effect to the surrounding nerves and tissues, thus reducing pain.

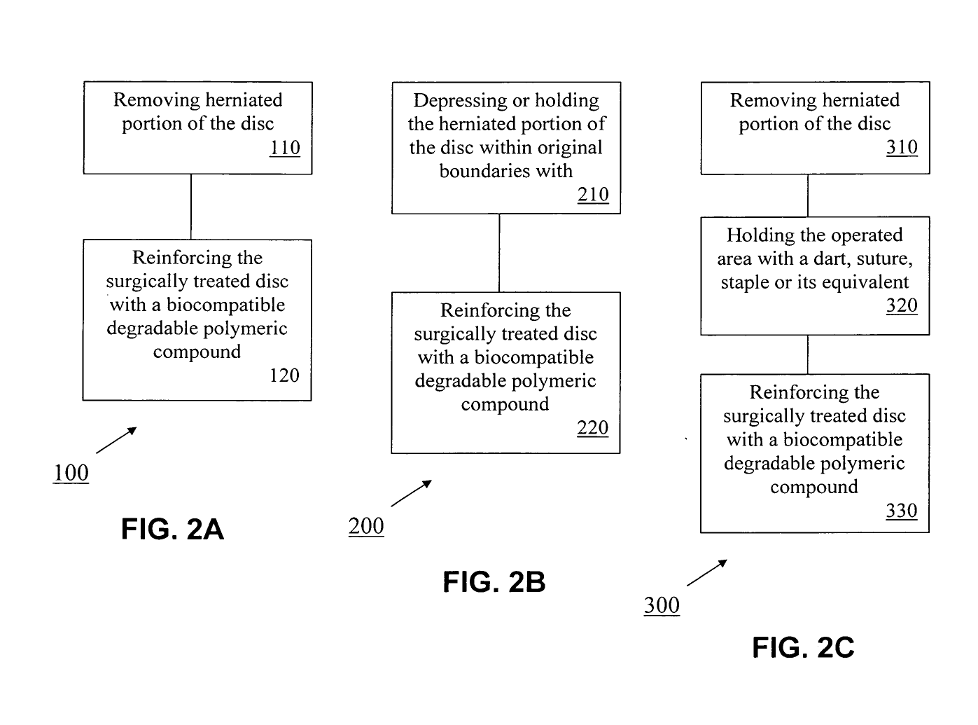

In one embodiment, a method 100 comprises the steps of surgically removing (110) a herniated portion of disc annulus from a herniated disc, and re-enforcing (120) the surgically treated disc with a biocompatible degradable polymeric compound (FIG. 2A).

The surgical procedure for removing the herniated disc material that presses on a nerve root or the spinal cord is called a “discectomy” or “partial discectomy.” Discectomy is well known to those skilled in the art. Discectomy can be done under either local, spinal or general anesthesia. During a conventional discectomy (also called “open discectomy”), the patient lays face down on the operating table, generally in a kneeling position. A small incision is made in the skin over the herniated disc and the muscles over the spine are pulled back from the bone. A small amount of bone may be removed so the surgeon can see the compressed nerve. The herniated disc and any loose pieces are removed until the disc no longer presses on the nerve. Any bone spurs (osteophytes) are also taken out to make sure that the nerve is free of pressure. Usually, there is very little bleeding.

Discectomy may also be performed percutaneously using specially designed instruments. Percutaneous means “through the skin” or using a very small incision. Percutaneous discectomy is different from conventional open discectomy or microdiscectomy. There are several percutaneous procedures. All of them involve inserting small instruments between the vertebrae in order to gain access to the disc, typically from the posterior side of the patient. X-ray monitoring is used during surgery to guide the movement of the surgical instruments. The surgeon can remove disc tissue by cutting it out, scraping in out, or suctioning it out of the disc, or by using lasers, RF or electrical devices to burn, shrink or evaporate the disc material.

After discectomy, a biocompatible degradable polymeric compound is injected into the operated area of the disc, both inside and outside the disc, to reinforce the annulus wall and deliver a matrix that will promote an escalated healing response at the site. The biocompatible degradable polymeric compound can be injected immediately after the discectomy or in a follow-up procedure days, weeks, or months after the discectomy. The injection time is determined by the attending physician based on the nature and extent of the discectomy, the condition of the herniated disc, and other patient concerns.

Examples of the biocompatible degradable polymeric compound include, but are not limited to, fibrin, type I collagen, type II collagen, type III collagen, fibronectin, laminin, hyaluronic acid (HA), hydrogel, pegylated hydrogel, chitosan, and combinations thereof.

In a preferred embodiment, the biocompatible degradable polymeric compound is a fibrin sealant. The fibrin sealant is formed from fibrinogen and an activating agent that converts fibrinogen to fibrin. Fibrinogen can be autologous (i.e., from the patient to be treated), heterologous (i.e., from other human, pooled human supply, or non-human sources such as bovine and fish), or recombinant. Fibrinogen can be fresh or frozen. Fibrinogen is commercially available in freeze-dried form. Freeze-dried fibrinogen is typically reconstituted in a solution containing aprotinin (a polyvalent protease inhibitor which prevents premature degradation of the formed fibrin). In one embodiment, the reconstitution solution contains aprotinin at a concentration of 3000 KIU/ml. Typical concentrations for aprotinin range between 2000-4000 KIU/ml

The activating agent can be any agent that causes fibrinogen to form fibrin. Examples of the activating agent include, but are not limited to, thrombin and thrombin-like enzymes. Thrombin is an enzyme that converts fibrinogen to fibrin. Thrombin can be autologous (i.e., from the patient to be treated), heterologous (i.e., from other human, pooled human supply, or non-human source such as bovine, fish, various arachnids and other venomous species), or recombinant. Thrombin can be fresh or frozen. Thrombin is commercially available in freeze-dried form. Freeze-dried thrombin can be reconstituted in water or water containing calcium ions. In one embodiment, the reconstitution solution contains calcium chloride in the range of about 1 to 100 mmol/ml.

A thrombin-like enzyme is any enzyme that can catalyze the formation of fibrin from fibrinogen. A common source of thrombin enzymes is from bovines. Another common source of thrombin-like enzymes is snake venom. Other sources of thrombin-like enzymes include various venomous marine life, such as jellyfish, sea snakes, cone shells, and sea urchins. Preferably, the thrombin-like enzyme is purified from the venom. Depending on the choice of thrombin-like enzyme, such thrombin-like enzyme can release fibrinopeptide A—which forms fibrin 1-fibrinopeptide B—which forms des BB fibrin—or both fibrinopeptide A and B—which forms fibrin II. Thrombin-like enzymes that release fibrinopeptide A and B may do so at different rates. Thus, the resultant composition could be, for example, a mixture of fibrin II and fibrin I or a mixture of fibrin II and des BB fibrin.

TABLE I is a nonlimiting list of the sources of the snake venoms that can be used with the herein disclosed methods, the name of the thrombin-like enzyme, and which fibrinopeptide(s) is released by treatment with the enzyme.

TABLE 1

| | | Fibrinopeptide |
|---|---|---|
| Source | Name | Released |
| Agkistrodon acutus | Acutin | A |
| A. contortrix contortrix | Venzyme | B, (A)* |
| A. halys pallas | | B, (A)* |
| A. (Calloselasma) | Ancrod, Arvin | A |
| rhodostoma | | |
| Bothrops asper | Asperase | A |
| B. atrox, B. moojeni, | Batroxobin | A |
| B. maranhao | | |
| B. insularis | Reptilase | A, B |
| B. jararaca | Botropase/bothrombin | A |
| Lachesis muta muta | Defibrase | A, B |
| Crotalus adamanteus | Crotalase | A |
| C. durissus terrificus | | A |
| Trimeresurus flavoviridis | Flavoxobin/habutobin | A |
| T. gramineus | Grambin | A |
| Bitis gabonica | Gabonase | A, B |

*( ) means low activity.

For a review of thrombin-like enzymes from snake venoms, see H. Pirkle and K. Stocker, Thrombosis and Haemostasis, 65(4):444-450 (1991). The preferred thrombin-like enzymes are Batroxobin, especially from B. moojeni, B. maranhao and B. atrox; and Ancrod, especially from A. rhodostoma.

In the herein disclosed methods, fibrin formation begins immediately on contact of the fibrinogen and the activating agent, such as in the Y-connector of a dual syringe injection device. One such dual syringe injection device is described in U.S. Patent Application Ser. No. 60/854,413, which is hereby incorporated by reference in its entirety.

The term “injecting” fibrin sealant as used herein thus encompasses any injection of components that form fibrin in the disc, including circumstances where a portion of the components react to form fibrin due to mixing prior to contact with or actual introduction into the disc. The herein disclosed methods include the sequential injection of the components of the fibrin sealant into the disc, such as by injecting the activating agent followed by the fibrinogen, or by injecting the fibrinogen followed by the activating agent. Likewise, the fibrinogen and the activating agent each can be intermittently injected into the disc.

Fibrin sealants mimic the final stage of the natural clotting mechanism. Fibrin sealants also provide a natural biologic matrix that promotes the healing process. Typically, such sealants entail the mixing of a fibrinogen component with an activating enzyme such as thrombin. To increase biocompatibility of the sealant with host tissue, various components may be supplied endogenously from host body fluids. Combining the reconstituted components produces a viscous solution that quickly sets into an elastic coagulum. A method of preparing a conventional fibrin sealant is described by J. Rousou, et al. (J. Rousou, et al. Journal of Thoracic and Cardiovascular Surgery, 1989, 97:194-203). Cryoprecipitate derived from source plasma is washed, dissolved in a buffer solution, filtered and freeze-dried. The freeze-dried fibrinogen is reconstituted in solution containing a fibrinolysis inhibitor. The solution is stirred and heated to a temperature of about 37° C. Each solution (the thrombin and fibrinogen solutions) is drawn up in a syringe and mounted on a Y-connector to which a needle is attached for delivery of the combined solution (see, e.g. the Duploject™ device, from ImmunoAG, Vienna, Austria). Thus, mixing of the components only occurs during the delivery process, which facilitates clot formation only at the desired site of application. The components should be injected sufficiently quickly to avoid the passage becoming blocked due to fibrin formation in the needle and/or Y-connector.

In one embodiment, a dual-syringe injector is used and the mixing of the fibrin sealant components at least partially occurs in the Y-connector and in the needle mounted on the Y-connector, with the balance of the clotting occurring in the disc. This method of preparation facilitates the formation of a fibrin clot at the desired site in the disc during delivery, or immediately thereafter.

In another embodiment, freeze-dried fibrinogen is reconstituted to a concentration of about 75-115 mg/ml, and freeze-dried thrombin is reconstituted separately to a final concentration of about 400-600 I.U./ml, preferably in a concentration of about 1-10 I.U./ml and more preferably at about 4-5 I.U./ml. Freeze-dried fibrinogen and freeze-dried thrombin are available in kit form from manufacturers such as Baxter under names such as TISSEEL™. These two fibrin sealant components can be prepared in about 2 ml samples each to yield approximately 4 ml of total sealant (reconstituted fibrinogen plus reconstituted thrombin). In another embodiment, at least one of the reconstituted fibrinogen and thrombin is reconstituted using a solution containing at least one additive. A preservative-free reconstituting solution may be used, but is not required.

The point, or points, of injection (e.g., at the tip of a spinal needle) can be in the nucleus pulposus, within the annulus fibrosus, or outside the annulus fibrosus. If the injection occurs in the nucleus pulposus, the injected components may form a patch at the interface between the nucleus pulposus and the annulus fibrosus, or, more commonly, the components flow into the defect(s) (e.g., fissures) of the annulus fibrosus and potentially “overflow” into the interdiscal space. Over-pressurizing the disc when injecting the components into the disc should be avoided.

The fibrin sealant may be administered with an anesthetic, such as a local anesthetic. Representative local anesthetics include but are not limited to lidocaine HCL (often sold in concentrations of 1.5 percent or 4 percent), SARAPIN anesthetic (a sterile aqueous solution of soluble salts and bases from Sarraceniaceae (Pitcher Plant), and bupivacaine HCL (also known as marcaine, which is often sold in concentrations of 0.5 percent and 0.75 percent). The chemical name for lidocaine is alpha-diethylaminoaceto-2,6-xylidide, and the IUPAC name is 2-(diethylamino)-N-(2,6-dimethylphenyl)acetamide. The chemical name for bupivicaine is 1-butyl-N-(2,6-dimethylphenyl)-2-piperidinecarboxamide, sometimes referred to as 1-butyl-2′,6′-pipecoloxylidide monohydrochloride, having registry number 14252-80-3. Alternatively, procaine (2-diethylaminoethyl 4-aminobenzoate hydrochloride) or other local anesthetic can be employed. Among the local anesthetics, bupivacaine is preferred. Combinations of anesthetics also can be used. The anesthetic can be injected with the fibrin sealant. Alternatively, the anesthetic can be injected separately, either before or after the injection of the fibrin sealant. Preferably, the anesthetic is injected prior to, or simultaneously with, the injection of the fibrin sealant.

In one embodiment, a solution containing a local anesthetic is used to reconstitute the fibrinogen or the activating agent. In another embodiment, the fibrinogen or the activating agent is reconstituted without an anesthetic, and the anesthetic is then added to the reconstituted fibrinogen or the activating agent.

In general, the amount of anesthetic used should be chosen so as to be effective in alleviating the pain of injection when the sealant is injected or otherwise introduced into the disc. In one embodiment, a solution containing about 0.1 to about 10 percent by weight of anesthetic is used. The injected volume of the anesthetic solution can vary widely, such as from about 0.1 ml to about 5 ml, depending one the mode of injection.

The fibrin sealant may be administered with one or more additives. As used herein, the term “additives” includes antibiotics; antiproliferative, cytotoxic, and antitumor drugs including chemotherapeutic drugs; analgesic; antiangiogen; antibody; antivirals; cytokines; colony stimulating factors; proteins; chemoattractants; chelating agent such as EDTA; histamine; antihistamine; erythropoietin; antifungals; antiparasitic agents; non-corticosteroid anti-inflammatory agents; anticoagulants; anesthetics including local anesthetics such as lidocaine and bupivicaine; analgesics; oncology agents; cardiovascular drugs; vitamins and other nutritional supplements; hormones; glycoproteins; fibronectin; peptides including polypeptides and proteins; interferons; cartilage inducing factors; protease inhibitors; vasoconstrictors, vasodilators, demineralized bone or bone morphogenetic proteins; hormones; lipids; carbohydrates; proteoglycans such as aggrecan (chondrotin sulfate and deratin sulfate), versican, decorin, and biglycan; antiangiogenins; antigens; deminerized bone matrix (DBM); hyaluronic acid and salts and derivatives thereof; polysaccharides; cellulose compounds such as methyl cellulose, carboxymethyl cellulose, and hydroxy-propylmethyl cellulose and derivatives thereof; antibodies; gene therapy reagents; genetically altered cells, stem cells including mesenchymal stem cells with transforming growth factor, and/or other cells; cell growth factors to promote rehabilitation of damaged tissue and/or growth of new, healthy tissue such as BMP7 and BMP2; type I and II collagen; elastin; sulfated glycosaminoglycan (sGAG), glucosamine sulfate; pH modifiers; methylsulfonylmethane (MSM); osteogenic compounds; osteoconductive compounds; plasminogen; nucleotides; oligonucleotides; polynucleotides; polymers; osteogenic protein 1 (OP-1 including recombinant OP-1); LMP-1 (Lim Mineralization Protein-1); cartilage including autologous cartilage; oxygen-containing components; enzymes such as, for example, peroxidase, which mediate the release of oxygen from such components; melatonin; vitamins; and nutrients such as, for example, glucose or other sugars. In one embodiment, the additive is a growth factor that promotes rehabilitation of the damaged tissues.

Any of the aforementioned additives may be added to the fibrin sealant separately or in combination. For example, one or more of these additives can be injected with the fibrin sealant. Combinations of these additives can be employed and different additives can be used in the solutions that are used to reconstitute the fibrinogen or the activating agent. In one embodiment, a solution containing a local anesthetic and glucosamine sulfate is used to reconstitute the fibrinogen, and a solution containing type II collagen is used to reconstitute the activating agent. Likewise, one or more of these additives can be injected be injected separately, either before or after the injection o the fibrin sealant.

For solutions containing an incompletely water-soluble additive(s), an anti-caking agent such as polysorbate, may be added to facilitate suspension of this component.

The fibrin sealant will generally be used in an amount effective to achieve the intended result, i.e., reinforce the annulus wall weakened by the surgical procedure and promote an escalated healing response at the site. The effective amount of the fibrin Sealant administered will depend upon a variety of factors, including, for example, the type of procedure used to remove the herniated portion of the disc, the site of the surgical procedure, the mode of administration, whether the desired benefit is prophylactic or therapeutic, the severity of the indication being treated and the age and weight of the patient, and the bioavailability of the particular active agent. Determination of an effective dosage is well within the capabilities of those skilled in the art.

Effective dosages may be estimated initially from in vitro assays and in vivo animal models. Suitable animal models of degenerative disc diseases and discogenic pain include rat and rabbit models described in, for example, Norcross et al. An in vivo model of degenerative disc disease, J. Orthopaedic Research, 2003, 21:183-188; and Larson et al., Biologic Modification of Animal Models of Intervertebral Disc Degeneration, The Journal of Bone and Joint Surgery (American), 2006, 88:83-87. Ordinarily, skilled artisans can routinely adapt such information to determine dosages suitable for human administration.

The fibrinogen is typically used in a concentration range of 50-150 mg/ml. The amount of activating agent such as thrombin can be varied to reduce or lengthen the time to complete fibrin formation. The fibrinogen is typically in the range 50 to 150 mg/ml and the thrombin is typically in the range 4 IU/ml to 600 IU/ml. In general, the higher level of thrombin per unit amount of fibrinogen, the faster fibrin formation occurs. If slower fibrin formation is desired, then less thrombin is used per unit fibrinogen. The fibrin formation time (i.e., the polymerization time of the fibrinogen) may be important for controlling the time at which the clot forms so as to ensure the fibrin sealant sets up at the proper site and time in the body rather than setting-up prematurely. Additionally, the aggressiveness of the mixing of the components plays a significant role in the setting time. The method of delivery can have a significant effect on clot time, uniformity of mixing density and strength of clot. Likewise, varying the fibrinogen concentration may change the density of the combined components, which may be important for controlling flow through a long conduit such as a catheter into the body. The use of calcium ions (such as from calcium chloride) in the thrombin component solution will affect the strength of the fibrin so formed, with increasing amounts of calcium ions increasing the strength of the fibrin clot.

For intradiscal injections, the total volume of the injection is limited. Typically, a total volume of 1-5 ml fibrin sealant is used for intradiscal injections. A larger volume of the fibrin sealant may be applied extradiscally.

The dosage and volume of fibrin sealant may be adjusted individually to provide local concentrations of the agents that are sufficient to maintain a therapeutic effect. For example, the fibrin sealant may be administered in a single injection or by sequential injections. The injection may be repeated periodically. Skilled artisans will be able to optimize effective local dosages and the injection regimen without undue experimentation.

Preferably, the fibrin sealant will provide a therapeutic benefit without causing substantial toxicity. Toxicity of the fibrin sealant may be determined using standard pharmaceutical procedures. The dose ratio between toxic and therapeutic effect is the therapeutic index. Agents that exhibit high therapeutic indices are preferred.

Preferably, a non-iodinated contrast agent may be used in conjunction with the injection of the fibrin sealant to ensure the correct placement at the site and avoidance of blood vessels. The contrast agent may be injected prior to injection of the fibrin sealant. Alternatively, the contrast agent may be included in the fibrinogen component or the activating agent component that is injected into the disc. Contrast agents and their use are well known to those skilled in the art.

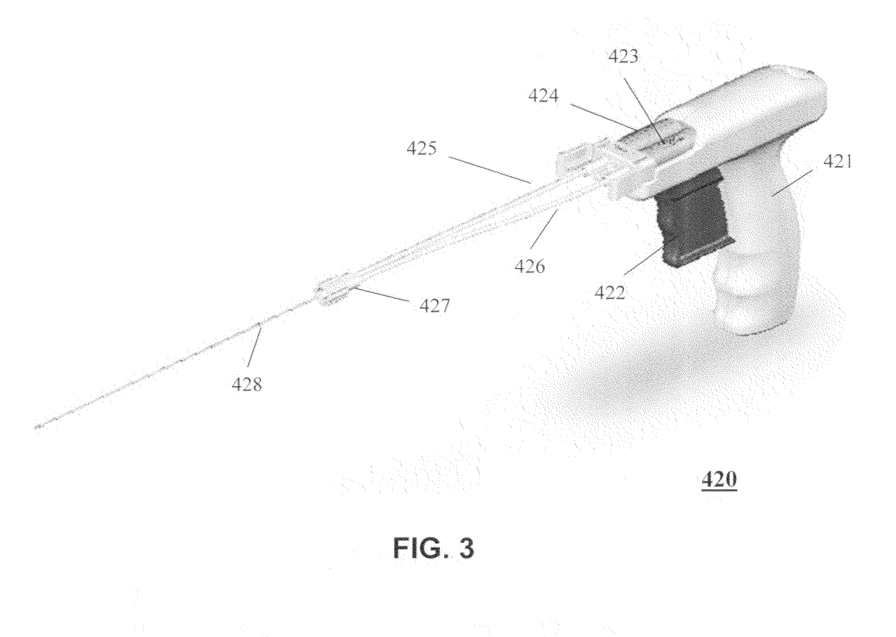

The fibrin sealant may be injected into or outside the disc using a delivery device such as that shown in FIG. 3 Delivery device 420 includes main housing 421 into which are inserted fibrinogen capsule 423 and thrombin capsule 424. Trigger 422, in conjunction with a pressure monitor (not shown) controls injection of the fluids. Attached to the capsules 423, 424 is an inner needle assembly including delivery tubes 425 and 426. Connector 427 serves to connect the delivery tubes 425, 426 to intradiscal needle 428.

In another embodiment, a method (200) comprises the steps of surgically depressing and holding (210) a herniated portion of disc annulus within the original boundaries of said herniated disc and held within the original boundaries by a dart, arrow, suture, staple or its equivalent, followed by re-enforcing (220) the surgically treated disc with a biocompatible degradable polymeric compound (FIG. 2B).

Briefly, a telescoping catheter or stacking catheter is inserted percutaneously until it reaches the herniated disc. Specially designed bent tonsil forceps or a similar device is then inserted through the catheter and into the herniated area of the annulus to pinch and depress the herniation back into the disc and away from the spinal cord or depressed nerve structures that are generating pain. A specially designed tool is then inserted through the catheter and a dart, arrow, suture, staple or its equivalent is placed across the weakened annular margins of the annulus. The dart, arrow, suture, staple or its equivalent would pinch the weakened portion of the annulus and compress the hernia, holding the herniated annulus within the original boundaries of the disc. Finally, a biocompatible degradable polymeric compound, such as a fibrin sealant, is injected directly into the disc nucleus, the repaired annulus area and around the exterior of the herniated annulus site to provide a temporary reinforcing “patch” or “glue patch” to the annulus. The biocompatible degradable polymeric compound will promote an escalated healing response at the site and would also form a natural biologic protective sheath over the exposed nerve endings which may also be enflamed from leaking nucleus pulposus. Preferably, the dart, arrow, suture, staple or its equivalent is made of a resorbable material.

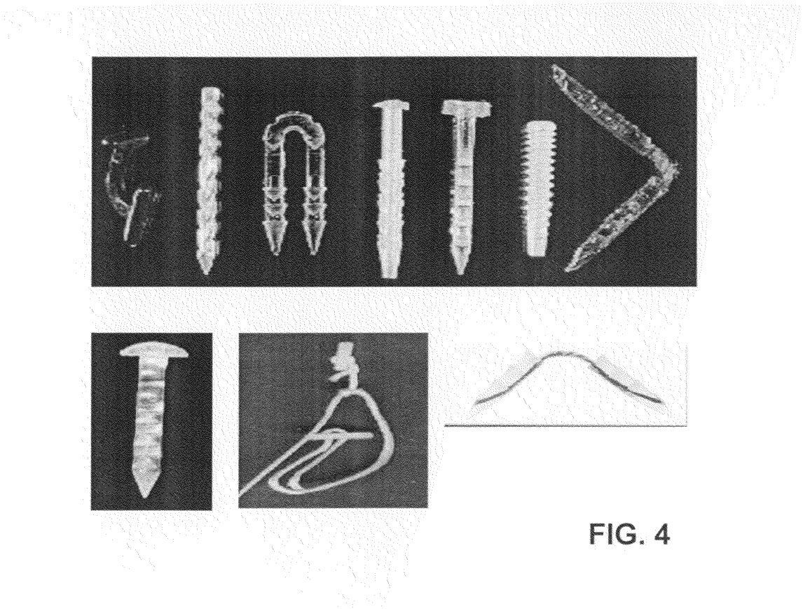

Resorbable materials for sutures, arrows, darts and staples are well known in the minimally invasive surgery fields of orthopedics, sports medicine, endoscopic, and general surgery. Examples of the resorbable materials include, but are not limited to, catgut, siliconized catgut (SICAT), chromic catgut, polyglycolic acid (PGA), polylactic acid (PLA), and copolymers of PGA/PLA. FIG. 4 shows pictures of some commercially available resorbable arrows, darts and staples that can be used in the method 200.

In yet another embodiment, a method (300) comprises the steps of surgically removing (310) the herniated portion of the disc annulus by discectomy, holding (320) the disc annulus within the original boundaries by a dart, suture, staple or its equivalent, and re-enforcing (330) the surgically treated disc with a biocompatible degradable polymeric compound to prevent re-herniation (FIG. 2C).

In all the above-described embodiments, the biocompatible degradable polymeric compound can be injected immediately after the surgical procedure or in a follow-up procedure days, weeks, or months after the surgical procedure. Preferably, the biocompatible degradable polymeric compound is fibrin sealant.

The disclosed method may be better understood by reference to the following examples, which are representative and should not be construed to limit the scope of the claims hereof.

### EXAMPLES

### Example 1

### Percutaneous Annulus Hernia Decompression Combined with a Later Injection of Fibrin Sealant

A 51-year old female patient suffering from a herniated L5/S1 disc with degenerative disc disease received a partial disc decompression with a Stryker DeKompressor® (Stryker Instruments, Kalamazoo, Mich.). Patient received limited relief for symptoms of radiculopathy, (extremity pain and weakness), but continued to suffer discogenic pain (pain in the back, emanating from the disc). The patient was subsequently diagnosed with induced leaking disc syndrome. The patient received a follow-up fibrin sealant treatment four months after the surgery. Fibrin sealant was injected both internally and externally at the disc site of the decompression. The patient received approximately 2 ml of fibrin sealant intradiscally and an additional 1-2 ml of fibrin sealant extradiscally, for a total treatment of approximately 4 ml. The patient has been doing well since the procedure. She received an epidural steroid injection in the same region two years after the procedure and is still doing well.

### Example 2

### Percutaneous Annulus Hernia Decompression Combined with Immediate Injection of Fibrin Sealant

A 66-year old male patient suffering from a herniated L4/L5 disc with degenerative disc disease and internal disc disruptions in discs L2/L3 & L3/L4 received a partial disc decompression with a Stryker DeKompressor® (Stryker Instruments, Kalamazoo, Mich.). The patient was treated with fibrin sealant immediately on the day of surgery. A total of 5 ml of fibrin sealant was injected. Approximately 3 ml of fibrin sealant was injected into the L4/L5 disc and around the exterior surgical site, Approximately 2 ml of fibrin sealant was injected into the nucleus of the L2/L3 & L3/L4 discs (1 ml for each disc). The patient has had good result for more than two years since the procedure, with no further treatment needed.

### Example 3

### Injection of Fibrin Sealant with a Dual-Syringe Injector

Injection of the fibrin sealant involves several steps, which are outlined below. The example presented is based on use of the delivery device 120 shown in FIG. 2A.

### Pre-Medication

As a first step, intravenous antibiotics are administered 15 to 60 minutes prior to commencing the procedure as prophylaxis against discitis. Patients with a known allergy to contrast medium should be pre-treated with H1 and H2 blockers and corticosteroids prior to the procedure in accordance with International Spine Intervention Society (ISIS) recommendations. Sedative agents may be administered but the patient should remain awake during the procedure and capable of responding to pain from pressurization of the disc. If the fibrin sealant is injected immediately after a surgical procedure (e.g., discectomy), the pre-medication step may not be necessary.

### Preparation

The injection procedure should be performed in a suite suitable for aseptic procedures and equipped with fluoroscopy (C-arm or two-plane image intensifier) and an x-ray compatible table to allow visualization of needle placement.

Local anesthetic for infiltration of skin and deep tissue and nonionic contrast medium with 10 mg per cc of antibiotic should be available for this procedure.

### Preparation of the Fibrin Sealant

Preparation of the fibrin sealant will require approximately 25 minutes. In an embodiment, freeze-dried fibrinogen and thrombin are reconstituted in a fibrinolysis inhibitor solution and a calcium chloride solution, respectively. The reconstituted fibrinogen and thrombin solutions are then combined upon delivery with the delivery device 120 to form the fibrin sealant within the treated disc.

### Preparation of the Delivery Device

Maintaining a sterile environment, the delivery device 120 is assembled and checked for function in preparation for the reconstituted thrombin and fibrinogen component solutions to be transferred into the device.

### Patient Positioning and Skin Preparation

The patient should lie on a radiography table in either a prone or oblique position depending on the physician's preference. By means of example for a lumbar disc treatment, the skin of the lumbar and upper gluteal region should be prepared as for an aseptic procedure using non-iodine containing preparations.

### Target Identification

For intradiscal injections, disc visualization and annulus fibrosus puncture should be conducted according the procedures used for provocation discography. The targeted disc should be approached from the side opposite of the patient's predominant pain. If the patient's pain is central or bilateral, the target disc can be approached from either side.

An anterior-posterior (AP) image of the lumbar spine is obtained such that the x-ray beam is parallel to the inferior vertebral endplate of the targeted disc. The beam should then be angled until the lateral aspect of the superior articular process of the target segment lies opposite the axial midline of the target disc. The path of the intradiscal needle should be parallel to the x-ray beam, within the transverse mid-plane of the disc, and just lateral to the lateral margin of the superior articular process.

### Placement of the Intradiscal Needle

The intradiscal needle is specifically designed to facilitate annular puncture and intradiscal access for delivery of the fibrin sealant. The intradiscal needle is manufactured with a slight bend in the distal end to enhance directional control of the needle as it is inserted through the back muscles and into the disc.

The intended path of the intradiscal needle is anesthetized from the subcutaneous tissue down to the superior articular process. The intradiscal needle initially may be inserted under fluoroscopic visualization down to the depth of the superior articular process. The intradiscal needle will be then slowly advanced through the intervertebral foramen while taking care not to impale the ventral ramus. If the patient complains of radicular pain or paraesthesia, advancement of the needle must be stopped immediately and the needle must be withdrawn approximately 1 cm. The path of the needle should be redirected and the needle slowly advanced toward the target disc. Contact with the annulus fibrosus will be noted as a firm resistance to continued insertion of the intradiscal needle. The needle will be then advanced through the annulus to the center of the disc. Placement of the needle is confirmed with both AP and lateral images. The needle tip should lie in the center of the disc in both views.

Once the needle position is confirmed, a small volume of non-ionic contrast medium may be injected into the disc. A minimal volume of contrast may be injected to insure avascular flow of the contrast media. If vascular flow is seen, the intradiscal needle should be repositioned and the contrast injection repeated.

### Loading the Delivery System

After correct placement of the intradiscal needle is confirmed, the reconstituted fibrinogen and thrombin solutions are transferred into the appropriate chambers of the delivery device 120.

### Attaching the Inner Needle Assembly and Intradiscal Needle

The inner needle assembly next is attached to the delivery device 120, and air is expelled from the device. The inner needle assembly is next inserted into the intradiscal needle which is already in the center of the target disc, creating a coaxial delivery needle.

### Delivery of the Fibrin Sealant

Placement of the intradiscal needle tip in the center of the target disc is reconfirmed with AP and lateral images. The trigger is then depressed to begin application of fibrin sealant to the disc. Pressure should be monitored constantly when squeezing the trigger. To prevent over-pressurization of the disc, pressure should not exceed 100 psi (6.8 atm) for a lumbar disc.

Each full compression of the trigger will deliver approximately 1 mL of the fibrin sealant to the disc. When the trigger is released, it automatically resets to the fully uncompressed position. Once all of the fibrin sealant has been delivered, the trigger will stop advancing.

Periodic images of the disc should be taken during application of the fibrin sealant to insure that the intradiscal needle has not moved from the center of the disc.

Application of the fibrin sealant to the disc should continue until one of the three following events occurs.
1. The total desired volume of the fibrin sealant is delivered to the disc, usually between 1-3 ml.
2. Continued application of the fibrin sealant would require pressures above 100 psi (6.8 atm).
3. The patient cannot tolerate continuation of the procedure.

After the application of the fibrin sealant is stopped, the intradiscal needle is carefully removed from the patient. Patient observation and vital signs monitoring will be performed for about 20-30 minutes following the procedure.

Extradiscal injection of the fibrin sealant (i.e., injection of fibrin sealant to the exterior of the weakened portion of the herniated disc) may also be carried out using procedures described above. An additional 1-3 ml of fibrin sealant, or the remaining amount available in the delivery device, should be delivered to the external area of the disc that had received surgical decompression. If appropriate, additional amounts of fibrinogen and thrombin may be (prepared and) loaded into the delivery device and delivered to the extradiscal area of the disc annulus.

### Example 4

### Percutaneous Annulus Hernia Decompression Combined with the Application of a Reinforcing Resorbable Dart, Arrow, Stable or Suture and Injection of Fibrin Sealant

A patient suffering from a herniated lumbar disc will receive a percutaneous annulus hernia decompression with a Stryker DeKompressor® (Stryker Instruments, Kalamazoo, Mich.). The physician will then use specially designed arthroscopic instruments to deliver a resorbable arrow or dart similar to that used for knee meniscus repair to the annulus. The arrow will be inserted into the annulus on one side of the surgically treated area and the other end will be inserted into the annulus on the other side of the surgically treated area where the herniation is removed, thus bridging the area and reinforcing the weakened annulus. Resorbable staples, sutures or comparable devices could satisfy the same purpose. The physician will then deliver approximately 2 ml of fibrin sealant intradiscally and an additional 1-2 ml of fibrin sealant extradiscally to the area around the surgical repair and reinforcing resorbable arrow, dart, staple or suture, for a total treatment of approximately 4 ml, as described in the previous example

A similar application of fibrin sealant and or reinforcing resorbable is envisioned following a limited microdiscectomy or partial nucleotomy.

The herein described methods may be used to address various conditions through use of the surgical procedure and fibrin sealant. The disclosure references particular means, materials and embodiments elaborating limited application of the claims. The claims are not limited to these particulars and applies to all equivalents. Although the claims make reference to particular means, materials and embodiments, it is to be understood that the claims not limited to these disclosed particulars, but extend instead to all equivalents.

What is claimed is:

1. A method of treating a herniated disc, comprising:
surgically removing a herniated portion of a disc annulus from said herniated disc; and
re-enforcing the surgically treated disc with an effective amount of a biocompatible degradable polymeric compound.

2. The method of claim 1, wherein said re-enforcing step comprises injecting said biocompatible degradable polymeric compound at a surgically treated disc area.

3. The method of claim 2, wherein said re-enforcing step comprises injecting said biocompatible degradable polymeric compound into said surgically treated disc area.

4. The method of claim 2, wherein said re-enforcing step comprises injecting said biocompatible degradable polymeric compound into and outside said surgically treated disc area.

5. The method of claim 1, wherein said biocompatible degradable polymeric compound is a fibrin sealant

6. The method of claim 1, wherein said herniated portion of a disc annulus is removed by a procedure selected from the group consisting of conventional open discectomy, mini-open discectomy, and percutaneous discectomy.

7. The method of claim 5, wherein said fibrin sealant is injected immediately after the removal of said herniated portion of a disc annulus.

8. The method of claim 5, wherein said fibrin sealant is produced by mixing fibrinogen with an activating agent during injection.

9. The method of claim 8, wherein said activating agent is thrombin or a snake venom derivative.

10. The method of claim 8, wherein said fibrinogen is autologous fibrinogen.

11. The method of claim 8, wherein said fibrinogen is heterologous fibrinogen.

12. The method of claim 5, wherein said fibrin sealant is injected with an anesthetic.

13. The method of claim 5, wherein said fibrin sealant is injected with at least one additive.

14. The method of claim 13, wherein said at least one additive is selected from the group consisting of antibiotics; antiproliferative, cytotoxic, and antitumor drugs including chemotherapeutic drugs; analgesic; antiangiogen; antibody; antivirals; cytokines; colony stimulating factors; proteins; chemoattractants; EDTA; histamine; antihistamine; erythropoietin; antifungals; antiparasitic agents; non-corticosteroid anti-inflammatory agents; anticoagulants; anesthetics; analgesics; oncology agents; cardiovascular drugs; vitamins and other nutritional supplements; hormones; glycoproteins; fibronectin; peptides including polypeptides and proteins; interferons; cartilage inducing factors; protease inhibitors; vasoconstrictors, vasodilators, demineralized bone or bone morphogenetic proteins; hormones; lipids; carbohydrates; proteoglycans; antiangiogenins; antigens; demineralised bone matrix (DBM); hyaluronic acid and salts and derivatives thereof; polysaccharides; cellulose compounds and derivatives thereof; antibodies; gene therapy reagents; genetically altered cells, stem cells including mesenchymal stem cells with transforming growth factor, and/or other cells; cell growth factors; type II collagen; elastin; sulfated glycosaminoglycan (sGAG), glucosamine sulfate; pH modifiers; methylsulfonylmethane (MSM); osteogenic compounds; osteoconductive compounds; plasminogen; nucleotides; oligonucleotides; polynucleotides; polymers; osteogenic protein 1 (OP-1 including recombinant OP-1); LMP-1 (Lim Mineralization Protein-1); cartilage; oxygen-containing components; enzymes; melatonin; vitamins; and nutrients.

15. The method of claim 5, wherein said fibrin sealant in injected in the presence of aprotinin and calcium ions.

16. The method of claim 5, wherein said injection of said fibrin sealant is performed using a dual syringe injector.

17. The method of claim 16, wherein said injecting step comprises:
inserting an introducer needle having a tip into a position adjacent to or inside a surgically treated disc area;
inserting a second needle or a polymeric catheter through the introducer needle up to but not beyond the tip of the introducer needle; and
simultaneously injecting a first fibrin sealant component through the introducer needle and a second fibrin sealant component through the second needle or polymeric catheter,
wherein said first fibrin sealant component is fibrinogen and said second fibrin sealant component is an activating agent, or
wherein said first fibrin sealant component is an activating agent and said second fibrin sealant component is fibrinogen.

18. The method of claim 17, wherein said injection is pressure-monitored.

19. The method of claim 1, wherein said herniated disc is a lumbar disc.

20. The method of claim 1, wherein said herniated disc is a cervical disc.

21. The method of claim 1, wherein said herniated disc is a thoracic disc.

22. The method of claim 1, further comprising injecting a contrast agent into said herniated disc before the injection of said biocompatible degradable polymeric compound.

23. A method of treating a herniated disc, comprising:
surgically depressing and holding a herniated portion of disc annulus within original boundaries of the herniated disc; and
re-enforcing the surgically treated disc with an effective amount of a biocompatible degradable polymeric compound.

24. The method of claim 23, wherein said re-enforcing step comprises injecting said biocompatible degradable polymeric compound at a surgically treated disc area.

25. The method of claim 24, wherein said re-enforcing step comprises injecting said biocompatible degradable polymeric compound into said surgically treated disc area.

26. The method of claim 24, wherein said re-enforcing step comprises injecting said biocompatible degradable polymeric compound into and outside said surgically treated disc area.

27. The method of claim 24, wherein said biocompatible degradable polymeric compound is a fibrin sealant.

28. The method of claim 23, wherein said herniated portion of disc annulus is depressed into original boundaries of said herniated disc with tonsil forceps or a similar device.

29. The method of claim 28, wherein said biocompatible degradable polymeric compound is injected immediately after said depressing step.

30. The method of claim 23, wherein said herniated portion of disc annulus is depressed into original boundaries of said herniated disc and is held in place with a dart, arrow, suture, staple or its equivalent.

31. The method of claim 30, wherein said dart, suture, staple or its equivalent is made of resorbable materials.

32. The method of claim 30, wherein said biocompatible degradable polymeric compound is injected immediately after the placement of said dart, arrow, suture staple or its equivalent.

33. The method of claim 27, wherein said fibrin sealant is produced by mixing fibrinogen with an activating agent during injection.

34. The method of claim 33, wherein said activating agent is thrombin or a snake venom derivative.

35. The method of claim 33, wherein said fibrinogen and said activating agent are injected with a dual syringe injector.

36. The method of claim 35, where said injection is pressure-monitored.

37. The method of claim 33, wherein said fibrinogen is autologous fibrinogen.

38. The method of claim 33, wherein said fibrinogen is heterologous fibrinogen.

39. The method of claim 27, wherein said fibrin sealant is injected with an anesthetic.

40. The method of claim 27, wherein said fibrin sealant is injected with at least one additive.

41. The method of claim 40, wherein said at least one additive is selected from the group consisting of antibiotics; antiproliferative, cytotoxic, and antitumor drugs including chemotherapeutic drugs; analgesic; antiangiogen; antibody; antivirals; cytokines; colony stimulating factors; proteins; chemoattractants; EDTA; histamine; antihistamine; erythropoietin; antifungals; antiparasitic agents; non-corticosteroid anti-inflammatory agents; anticoagulants; anesthetics; analgesics; oncology agents; cardiovascular drugs; vitamins and other nutritional supplements; hormones; glycoproteins; fibronectin; peptides including polypeptides and proteins; interferons; cartilage inducing factors; protease inhibitors; vasoconstrictors, vasodilators, demineralized bone or bone morphogenetic proteins; hormones; lipids; carbohydrates; proteoglycans; antiangiogenins; antigens; DBM; hyaluronic acid and salts and derivatives thereof; polysaccharides; cellulose compounds and derivatives thereof; antibodies; gene therapy reagents; genetically altered cells, stem cells including mesenchymal stem cells with transforming growth factor, and/or other cells; cell growth factors; type II collagen; elastin; sulfated glycosaminoglycan (sGAG), glucosamine sulfate; pH modifiers; methylsulfonylmethane (MSM); osteogenic compounds; osteoconductive compounds; plasminogen; nucleotides; oligonucleotides; polynucleotides; polymers; osteogenic protein 1 (OP-1 including recombinant OP-1); LMP-1 (Lim Mineralization Protein-1); cartilage; oxygen-containing components; enzymes; melatonin; vitamins; and nutrients.

42. The method of claim 27, wherein said fibrin sealant in injected in the presence of aprotinin and calcium ions.

43. The method of claim 27, wherein the injecting step comprises:
inserting an introducer needle having a tip into a position adjacent to or inside a surgically treated disc area;
inserting a second needle or a polymeric catheter through the introducer needle up to but not beyond the tip of the introducer needle; and
simultaneously injecting a first fibrin sealant component through the introducer needle and a second fibrin sealant component through the second needle or polymeric catheter,
wherein said first fibrin sealant component is fibrinogen and said second fibrin sealant component is an activating agent, or
wherein said first fibrin sealant component is an activating agent and said second fibrin sealant component is fibrinogen.

44. The method of claim 23, wherein said herniated disc is a lumbar disc.

45. The method of claim 23, wherein said herniated disc is a cervical disc.

46. The method of claim 23, wherein said herniated disc is a thoracic disc.

47. The method of claim 24, further comprising injecting a contrast agent into said herniated disc before the injection of said biocompatible degradable polymeric compound.

48. A method of treating a herniated disc, comprising:
(a) surgically removing a herniated portion of disc annulus from said herniated disc; (b) placing a dart, suture, staple or its equivalent in a disc area weakened by step (a), and
(c) re-enforcing the surgically treated disc with an effective amount of a biocompatible degradable polymeric compound.

49. The method of claim 48, wherein said re-enforcing step comprises injecting said biocompatible degradable polymeric compound at said surgically treated disc annulus area.

50. The method of claim 49, wherein said biocompatible degradable polymeric compound is a fibrin sealant.
